(19) United States
(12) Patent Application Publication
(10) Pub. No.: US 2011/0207742 A1
(43) Pub. Date: Aug. 25, 2011

(54) METHOD FOR REDUCTION, STABILIZATION AND PREVENTION OF RUPTURE OF LIPID RICH PLAQUE

(75) Inventors: Hideyuki Kobayashi, Saitama (JP); Yasunobu Yoshinaka, Saitama (JP); Kimiyuki Shibuya, Saitama (JP)

(73) Assignee: KOWA CO., LTD., Nagoya-shi (JP)

(21) Appl. No.: 13/096,477

(22) Filed: Apr. 28, 2011

Related U.S. Application Data
(62) Division of application No. 11/721,402, filed on Jun. 11, 2007.
(60) Provisional application No. 60/634,532, filed on Dec. 10, 2004.

(51) Int. Cl. A61K 31/497; A61P 9/10; A61P 7/02

(52) U.S. Cl. 514/253.09

(57) ABSTRACT

There is to provide is an agent for reduction of a lipid rich plaque, stabilization of a lipid rich plaque and/or prevention of rupture of a lipid rich plaque in an atherosclerotic lesion comprising an effective amount of 2-[4-[2-(benzimidazole-2-ylthio)ethyl]piperazin-1-yl]-N-[2,4-bis(methylthio)-6-methyl-3-p yridyl]acetamide (hereinafter, referred to as compound 1), its pharmaceutically acceptable salt or a hydrate thereof and Pitavastatin, and a pharmaceutically acceptable carrier, wherein the agent is intended to be simultaneously administered, or separately administered with interval of time to a patient in need thereof There is also to provide a method for reduction of a lipid rich plaque, stabilization of a lipid rich plaque and/or prevention of rupture of a lipid rich plaque in an atherosclerotic lesion, comprising simultaneously administering, or separately administering with interval of time an effective amount of the compound 1, its pharmaceutically acceptable salt or a hydrate thereof and an effective amount of Pitavastatin to a patient in need thereof.

### CROSS-REFERENCE TO RELATED APPLICATIONS

This is a divisional application of U.S. patent application Ser. No. 11/721,402, filed on Jun. 11, 2007, which is a 371 of International Application No. PCT/JP05/23088, filed on Dec. 9, 2005, which claims the benefit of priority from the prior U.S. Provisional Application No. 60/634,532 filed on Dec. 10, 2004, the entire contents of which are incorporated herein by references.

### TECHNICAL FIELD

The present invention relates to a method for reduction of a lipid rich plaque, stabilization of a lipid rich plaque, and prevention of rupture of a lipid rich plaque in an atherosclerotic lesion. Specifically, the present invention relates to a method for reduction, stabilization, and prevention of rupture of a lipid rich plaque, including simultaneously administering, or separately administering with interval of time an effective amount of 2-[4-[2-(benzimidazole-2-ylthio)ethyl]piperazin-1-yl]-N-[2,4-bis(methylthio)-6-methyl-3-p yridyl]acetamide, its pharmaceutically acceptable salt or a hydrate thereof and an effective amount of Pitavastatin to attain not only quantitative change such as a reduction in plaque area in a plaque lesion but also qualitative change such as inhibition of macrophage accumulation and an increase in collagen.

### BACKGROUND ART

In recent years, as a result of change in lifestyle associated with improvement in living standards, that is, as a result of consumption of high-calorie and high-cholesterol foods, insufficient exercise, obesity, stresses of complicated society, and aging of the population or the like, there has been a rapid increase in arteriosclerotic diseases. The risk factors of these arteriosclerotic diseases are classified into some groups: smoking, obesity, hypertension, hyperuricemia, diabetes, and hyperlipidemia. Among them, improvement in hyperlipidemia including hypertriglyceridemia, reduction in HDL (HDL: high-density lipoprotein), and elevation of LDL (LDL: low-density lipoprotein) has received attention. Particularly, a reduction of high cholesterol has been given priority as a target of drug therapy, and various treatments have been attempted therefor. Among them, as a therapeutic drug that is the most effective in treating hypercholesterolemia, a drug (statin) which inhibits an HMG-CoA (HMG-CoA: 3-hydroxy-3-methylglutaryl-coenzyme A) reductase that is a rate-limiting enzyme of cholesterol biosynthesis can be mentioned. Cholesterol-lowering therapy using statin has achieved concrete results in treatment of various arteriosclerotic diseases resulting from hyperlipemia, such as myocardial infarction and cerebral infarction. Particularly, in terms of the improvement in the five-year survival rate of patients, the effectiveness of statin against coronary artery diseases such as acute myocardial infarction has been demonstrated from the results of large-scale clinical trials such as 4S (Scandinavian Simvastatin Survival Study) and WOS (COPS) (West of Scotland Coronary Prevention Study) (see Circulation, 96(12): 4424-4430, 1997; N. Engl. J. Med., 317(20): 1301-1307, 1995; Lancet, 344(19): 1383-1389, 1994). As described above, although statin preparations as represented by Simvastatin (see U.S. Pat. No. 4,444,784) and Pravastatin (see U.S. Pat. No. 4,346,227) are certainly effective, the ratio of inhibition to the incidence of coronary artery disease events is only about 30% at most, which is by no means satisfactory in the field of medical care. Further, as a mechanism for expression of medical benefits by statin, it has been known that statin not only inhibits cholesterol biosynthesis in the body but also reduces cholesterol in the liver, which enhances the expression of LDL receptors, thereby promoting uptake of LDL from the blood by increased LDL receptors, thus resulting in reduction in total plasma cholesterol (TC). Therefore, in the case of homozygous or heterozygous patients lacking LDL receptors, for example, patients with familial hypercholesterolemia (FH), it cannot be expected that LDL cholesterol will be sufficiently reduced. Further, it has been found that combined administration of a fibrate drug and statin to patients with also hypertriglyceridemia induces rhabdomyolysis, which recently has led to discontinuation of marketing of Cerivastatin (see U.S. Pat. No. 5,177,080) because rhabdomyolysis is a serious side effect. Under the circumstances, it is considered that a drug which can cure arteriosclerosis by directly acting on an arteriosclerotic lesion is more attractive than a drug which exhibits an anti-arteriosclerotic effect due to a reduction in TC, and therefore there has been a demand for such a drug which directly acts on an arteriosclerotic lesion.

Meanwhile, a plaque, which is a primary atherosclerotic lesion is composed of a lipid core filled with cholesterol and cholesterol esters and a fibrous material called extracellular matrix. Particular, a lipid rich plaque that is rich in lipid and inflammatory cells such as macrophage and is covered with a thin fibrous coating is called an “unstable plaque”. Such a lipid rich plaque is likely to rupture. If a lipid rich plaque ruptures, the contents of the plaque are exposed to vascular flow, which promotes thrombus formation. It is believed that thrombus formation as a result of plaque rupture causes acute coronary syndrome (ACS) such as unstable angina pectoris, acute myocardial infarction, and ischemic sudden death (see N. Engl. J. Med., 326(4): 242-250, 1992). In fact, it has been known, as a result of investigation, that the culprit lesion in about 75% of patients who died of ACS is due to thrombus formation caused by plaque rupture (see Circulation, 92(3): 657-671, 1995). Further, culprit lesions of myocardial infarction in terms of the degree of vessel stenosis, that is, the size of a plaque, it has become clear that most of them are in vessels with a degree of stenosis of less than 50% (see Circulation, 92(3): 657-671, 1995). This fact indicates that the cause of plaque rupture depends on the quality of a plaque rather than the size of a plaque.

Further, it is suspected that matrix metalloprotease (MMP) secreted by a large amount of aggregated macrophages is directly involved in plaque rupture. In this case, it is believed that MMP disintegrates fibrous collagen, thereby thinning and weakening a fibrous coating (see Nature Med., 8(11): 1257-1262, 2002). Furthermore, it has been reported that macrophages express tissue factor, thereby promoting thrombus formation at rupture sites (see Ann. N.Y. Acad. Sci., 902: 140-152, 2000).

Therefore, it can be considered that prevention of rupture of an “unstable plaque” is important to deal with ACS. Examples of a method for preventing rupture of an “unstable plaque” include inhibition of macrophage functions, inhibition of macrophage accumulation itself, inhibition of disintegration of fibrous collagen, and increase in collagen content of a fibrous coating to reinforce the fibrous coating. Namely, it can be said that as a method for preventing or treating ACS, drug therapy for stabilizing a lipid rich plaque by inhibiting macrophage accumulation and increasing collagen is more preferred than that for regressing a plaque by reducing TC.

Under the circumstances, an acylcoenzyme A: cholesterol O-acyltransferase (ACAT) inhibitor has received attention as a cholesterol-lowering drug having an action mechanism different from that of statin.

There have been many reports about the effect of an ACAT inhibitor on plaque regression, but in all the reports, TC values are also significantly reduced. Therefore, it is doubtful whether an ACAT inhibitor has a direct effect on plaque reduction, thereby causing confusion in data interpretation of these reports (see Exp. Opin. Invest. Drugs, 4(5): 353-387, 1995; Drug Discovery Today, 3: 19-25, 1998).

However, recently, some ACAT inhibitors which exhibit the effect of regressing a plaque without affecting TC have been reported. For example, Japanese Patent Application Laid-open No. 2002-255808 discloses an ACAT inhibitor to be used as an agent for plaque regression, and WO 01/034127 discloses an ACAT inhibitor for inhibiting accumulation of macrophages in a plaque and reducing the expression of MMP. In both of the patent documents, although quantitative change such as a plaque-reducing effect is described, there is no description about qualitative change such as increase or decrease in fibrous collagen which is one of the important factors contributing to plaque stabilization (Circ. Res., 86(1): 101-108, 2000). WO 01/034127 describes the effect of the compound (Avasimibe, hereinafter referred to as CI-1011) on plaque reduction, but in WO 01/034127 there is no description suggesting that CI-1011 can stabilize a plaque through qualitative change such as increase in fibrous collagen.

As is clear from the above description, an ACAT inhibitor different from a conventional one, that is, an ACAT inhibitor which can independently prevent plaque rupture by decreasing the percentage of plaque area occupied by macrophages and increasing the percentage of plaque area occupied by collagen without affecting TC fluctuations has not yet been known.

Meanwhile, recently there have also been some reports about an attempt to lowering blood lipid additively and synergistically or improve an arteriosclerotic lesion by changing the composition of blood lipoprotein or reduce the area of lipid deposition through combined use of lipid-lowering agents having different action mechanisms. However, these reports do not also concretely describe plaque stabilization (see WO 97/16184, WO 01/22962, WO 02/20009, and Japanese Patent Application Publication No. 11-515025). Further, as a result of close investigation of TC values of registered patients in large-scale clinical trials, it has been found that the average TC value in the case of CARE (Cholesterol And Recurrent Events trial; N. Engl. J. Med., 335(14): 1001-1009, 1996) is 209 mg/dL, and the average TC value in the case of LIPID (Long-term Intervention with Pravastatin in Ischaemic Disease; N. Engl. J. Med., 339(19): 1349-1357, 1998) is 218 mg/dL. These average TC values are not so high. The results of the clinical trials for such patients suggest limits on the production of effect of TC-lowering therapy using a single drug, thereby raising expectations for additional effects obtained by combined administration with another drug. However, it is clear that such expectations are placed on the effect of indirectly stabilizing an unstable plaque by reducing plaque area due to TC reduction or by reducing macrophages and increasing fibrous collagen associated with a reduction in macrophages (Circulation, 97(24): 2433-2444, 1998). Therefore, there has been a demand for a combination of drugs which can directly induce plaque stabilization.

### Disclosure of Invention

Under the circumstances, the present inventors have intensively investigated, and as a result they have found that an ACAT inhibitor disclosed in Example 32 of WO 98/54153, i.e., 2-[4-[2-(benzimidazole-2-ylthio)ethyl]piperazin-1-yl]-N-[2,4-bis(methylthio)-6-methyl-3-p yridyl]acetamide (hereinafter, referred to as compound 1), its pharmaceutically acceptable salt or a hydrate thereof can directly act on a blood vessel wall to decrease the percentage of plaque area occupied by macrophages and increase the percentage of plaque area occupied by collagen without significantly affecting TC fluctuations, that is, the compound 1, its pharmaceutically acceptable salt or a hydrate thereof can stabilize a lipid rich plaque to prevent the rupture of a lipid rich plaque. In addition, they have also found that such an effect can be synergistically enhanced by combined use with Pitavastatin that is an HMG-CoA reductase inhibitor, which has led to the completion of the present invention. In this regard, it is to be noted that the compound 1, that is, a macrophage selective ACAT inhibitor and a production method thereof are disclosed in WO 98/54153, and the disclosure thereof is incorporated herein by reference. WO 98/54153 describes not only the compound to be used for treatment of hypercholesterolemia and atherosclerosis but also an agent for (selectively) inhibiting macrophage foam cell formation, but does not describe the effect of plaque stabilization at all. On the other hand, Pitavastatin is disclosed in Japanese Patent No. 2569746, U.S. Pat. No. 5,856,336, and EP 304063. It is known that Pitavastatin has significantly high bioavailability and a strong effect on lowering of TC.

The present invention provides a method for reduction, stabilization and/or prevention of rupture of a lipid rich plaque in an atherosclerotic lesion, characterized by simultaneously administering, or separately administering with interval of time an effective amount of the compound 1, its pharmaceutically acceptable salt or a hydrate thereof and an effective amount of Pitavastatin to a patient in need thereof.

Further, the present invention provides a method for reduction, stabilization and/or prevention of rupture of a lipid rich plaque in an atherosclerotic lesion, including administering an effective amount of the compound 1, its pharmaceutically acceptable salt or a hydrate thereof and an effective amount of Pitavastatin as a single pharmaceutical preparation containing these active ingredients to a patient in need thereof.

Furthermore, the present invention provides a pharmaceutical composition for reduction, stabilization and/or prevention of rupture of a lipid rich plaque in an atherosclerotic lesion, including an effective amount of the compound 1, its pharmaceutically acceptable salt or a hydrate thereof and Pitavastatin, and a pharmaceutically acceptable carrier, wherein the composition is intended to be simultaneously administered, or separately administered with interval of time to a patient in need thereof.

Moreover, the present invention provides a pharmaceutical composition for reduction, stabilization and/or prevention of rupture of a lipid rich plaque in an atherosclerotic lesion including an effective amount of the compound 1, its pharmaceutically acceptable salt or a hydrate thereof and Pitavastatin, and a pharmaceutically acceptable carrier, wherein the composition is intended to be administered as a single pharmaceutical preparation containing these active ingredients to a patient in need thereof.

Moreover, the present invention provides an agent for reduction, stabilization and/or prevention of rupture of a lipid rich plaque in an atherosclerotic lesion, including an effective amount of the compound 1, its pharmaceutically acceptable salt or a hydrate thereof and Pitavastatin, and a pharmaceutically acceptable carrier, wherein the agent is intended to be simultaneously administered, or separately administered with interval of time to a patient in need thereof.

Moreover, the present invention provides an agent for reduction, stabilization and/or prevention of rupture of a lipid rich plaque in an atherosclerotic lesion, including an effective amount of the compound 1, its pharmaceutically acceptable salt or a hydrate thereof and Pitavastatin, and a pharmaceutically acceptable carrier, wherein the agent is intended to be administered as a single pharmaceutical preparation containing these active ingredients to a patient in need thereof.

Moreover, the present invention provides the use of the compound 1, its pharmaceutically acceptable salt or a hydrate thereof and Pitavastatin for producing an agent for reduction, stabilization and/or prevention of rupture of a lipid rich plaque, which is intended for use in simultaneously administering, or separately administering with interval of time an effective amount of the compound 1, its pharmaceutically acceptable salt or a hydrate thereof and an effective amount of Pitavastatin.

Moreover, the present invention provides the use of the compound 1, its pharmaceutically acceptable salt or a hydrate thereof and Pitavastatin for producing an agent for reduction, stabilization and/or prevention of rupture of a lipid rich plaque, which is intended for use in administering an effective amount of the compound 1, its pharmaceutically acceptable salt or a hydrate thereof and an effective amount of Pitavastatin as a single pharmaceutical preparation.

Moreover, the present invention provides a method for preventing thrombus formation caused by rupture of a lipid rich plaque in an atherosclerotic lesion, characterized by simultaneously administering, or separately administering with interval of time an effective amount of the compound 1, its pharmaceutically acceptable salt or a hydrate thereof and an effective amount of Pitavastatin to a patient in need thereof.

Moreover, the present invention provides a method for preventing thrombus formation caused by rupture of a lipid rich plaque in an atherosclerotic lesion, characterized by administering an effective amount of the compound 1, its pharmaceutically acceptable salt or a hydrate thereof and an effective amount of Pitavastatin as a single pharmaceutical preparation containing these active ingredients to a patient in need thereof.

Moreover, the present invention provides a pharmaceutical composition for preventing thrombus formation caused by rupture of a lipid rich plaque in an atherosclerotic lesion, including an effective amount of the compound 1, its pharmaceutically acceptable salt or a hydrate thereof and Pitavastatin, and a pharmaceutically acceptable carrier, wherein the composition is intended to be simultaneously administered, or separately administered with interval of time to a patient in need thereof.

Moreover, the present invention provides a pharmaceutical composition for preventing thrombus formation caused by rupture of a lipid rich plaque in an atherosclerotic lesion, including an effective amount of the compound 1, its pharmaceutically acceptable salt or a hydrate thereof and Pitavastatin, and a pharmaceutically acceptable carrier, wherein the composition is intended to be administered as a single pharmaceutical preparation containing these active ingredients to a patient in need thereof.

Moreover, the present invention provides an agent for preventing thrombus formation caused by rupture of a lipid rich plaque in an atherosclerotic lesion, including an effective amount of the compound 1, its pharmaceutically acceptable salt or a hydrate thereof and Pitavastatin, and a pharmaceutically acceptable carrier, wherein the agent is intended to be simultaneously administered, or separately administered with interval of time to a patient in need thereof.

Moreover, the present invention provides an agent for preventing thrombus formation caused by rupture of a lipid rich plaque in an atherosclerotic lesion, including an effective amount of the compound 1, its pharmaceutically acceptable salt or a hydrate thereof and Pitavastatin, and a pharmaceutically acceptable carrier, wherein the agent is intended to be administered as a single pharmaceutical preparation containing these active ingredients to a patient in need thereof.

Moreover, the present invention provides the use of the compound 1, its pharmaceutically acceptable salt or a hydrate thereof and Pitavastatin for producing an agent for preventing thrombus formation caused by rupture of a lipid rich plaque, which is intended for use in simultaneously administering, or separately administering with interval of time an effective amount of the compound 1, its pharmaceutically acceptable salt or a hydrate thereof and an effective amount of Pitavastatin.

Moreover, the present invention provides the use of the compound 1, its pharmaceutically acceptable salt or a hydrate thereof and Pitavastatin for producing an agent for preventing thrombus formation caused by rupture of a lipid rich plaque, which is intended for use in administering an effective amount of the compound 1, its pharmaceutically acceptable salt or a hydrate thereof and an effective amount of Pitavastatin as a single pharmaceutical preparation.

Moreover, the present invention provides a prophylactic and/or therapeutic method for acute coronary syndrome, acute myocardial infarction, unstable angina pectoris, and/or peripheral arterial obstruction, characterized by simultaneously administering, or separately administering with interval of time an effective amount of the compound 1, its pharmaceutically acceptable salt or a hydrate thereof and an effective amount of Pitavastatin to a patient in need thereof.

Moreover, the present invention provides a prophylactic and/or therapeutic method for acute coronary syndrome, acute myocardial infarction, unstable angina pectoris, and/or peripheral arterial obstruction, characterized by administering an effective amount of the compound 1, its pharmaceutically acceptable salt or a hydrate thereof and an effective amount of Pitavastatin as a single pharmaceutical preparation containing these active ingredients to a patient in need thereof.

Moreover, the present invention provides a pharmaceutical composition for preventing and/or treating acute coronary syndrome, acute myocardial infarction, unstable angina pectoris, and/or peripheral arterial obstruction, including an effective amount of the compound 1, its pharmaceutically acceptable salt or a hydrate thereof and Pitavastatin, and a pharmaceutically acceptable carrier, wherein the composition is intended to be simultaneously administered, or separately administered with interval of time to a patient in need thereof.

Moreover, the present invention provides a pharmaceutical composition for preventing and/or treating acute coronary syndrome, acute myocardial infarction, unstable angina pectoris, and/or peripheral arterial obstruction, including an effective amount of the compound 1, its pharmaceutically acceptable salt or a hydrate thereof and Pitavastatin, and a pharmaceutically acceptable carrier, wherein the composition is intended to be administered as a single pharmaceutical preparation containing these active ingredients to a patient in need thereof.

Moreover, the present invention provides a prophylactic and/or therapeutic agent for acute coronary syndrome, acute myocardial infarction, unstable angina pectoris, and/or peripheral arterial obstruction, including an effective amount of the compound 1, its pharmaceutically acceptable salt or a hydrate thereof and Pitavastatin, and a pharmaceutically acceptable carrier, wherein the agent is intended to be simultaneously administered, or separately administered with interval of time to a patient in need thereof.

Moreover, the present invention provides a prophylactic and/or therapeutic agent for acute coronary syndrome, acute myocardial infarction, unstable angina pectoris, and/or peripheral arterial obstruction, including an effective amount of the compound 1, its pharmaceutically acceptable salt or a hydrate thereof and Pitavastatin, and a pharmaceutically acceptable carrier, wherein the agent is intended to be administered as a single pharmaceutical preparation containing these active ingredients to a patient in need thereof.

Moreover, the present invention provides the use of the compound 1, its pharmaceutically acceptable salt or a hydrate thereof and Pitavastatin for producing a prophylactic and/or therapeutic agent for acute coronary syndrome, acute myocardial infarction, unstable angina pectoris, and/or peripheral arterial obstruction, wherein the use of the compound 1 is intended for use in simultaneously administering, or separately administering with interval of time an effective amount of the compound 1, its pharmaceutically acceptable salt or a hydrate thereof and an effective amount of Pitavastatin.

Moreover, the present invention provides the use of the compound 1, its pharmaceutically acceptable salt or a hydrate thereof and Pitavastatin for producing a prophylactic and/or therapeutic agent for acute coronary syndrome, acute myocardial infarction, unstable angina pectoris, and/or peripheral arterial obstruction, wherein the use of the compound 1 for use in administering an effective amount of the compound 1, its pharmaceutically acceptable salt or a hydrate thereof and an effective amount of Pitavastatin as a single pharmaceutical preparation.

Moreover, the present invention provides an agent for enhancing the effect of the compound 1, its pharmaceutically acceptable salt or a hydrate thereof on reduction, stabilization and/or prevention of rupture of a lipid rich plaque in an atherosclerotic lesion, including statins, preferably Pitavastatin.

Moreover, the present invention provides a pharmaceutical composition for enhancing the effect of the compound 1, its pharmaceutically acceptable salt or a hydrate thereof on reduction, stabilization and/or prevention of rupture of a lipid rich plaque in an atherosclerotic lesion, including statins, preferably Pitavastatin and a pharmaceutically acceptable carrier.

Moreover, the present invention provides the use of statins, preferably Pitavastatin as an agent for enhancing the effect of the compound 1, its pharmaceutically acceptable salt or a hydrate thereof on reduction, stabilization and/or prevention of rupture of a lipid rich plaque in an atherosclerotic lesion.

Moreover, the present invention provides the use of statins, preferably Pitavastatin for producing an agent for enhancing the effect of the compound 1, its pharmaceutically acceptable salt or a hydrate thereof on reduction, stabilization and/or prevention of rupture of a lipid rich plaque in an atherosclerotic lesion.

Moreover, the present invention provides a method for enhancing the effect of the compound 1, its pharmaceutically acceptable salt or a hydrate thereof on reduction, stabilization and/or prevention of rupture of a lipid rich plaque, including administering an effective amount of statins, preferably Pitavastatin to a patient to which an effective amount of the compound 1, its pharmaceutically acceptable salt or a hydrate thereof has been administered.

Moreover, the present invention provides a method for reduction, stabilization and/or prevention of rupture of a lipid rich plaque by enhancing the effect of the compound 1, its pharmaceutically acceptable salt or a hydrate thereof on reduction, stabilization and/or prevention of rupture of a lipid rich plaque, including administering an effective amount of statins, preferably Pitavastatin to a patient to which an effective amount of the compound 1, its pharmaceutically acceptable salt or a hydrate thereof has been administered.

Moreover, the present invention provides an agent for enhancing the effect of the compound 1, its pharmaceutically acceptable salt or a hydrate thereof on prevention of thrombus formation caused by rupture of a lipid rich plaque in an atherosclerotic lesion, including statins, preferably Pitavastatin.

Moreover, the present invention provides a pharmaceutical composition for enhancing the effect of the compound 1, its pharmaceutically acceptable salt or a hydrate thereof on prevention of thrombus formation caused by rupture of a lipid rich plaque in an atherosclerotic lesion, including statins, preferably Pitavastatin and a pharmaceutically acceptable carrier.

Moreover, the present invention provides the use of statins, preferably Pitavastatin as an agent for enhancing the effect of the compound 1, its pharmaceutically acceptable salt or a hydrate thereof on prevention of thrombus formation caused by rupture of a lipid rich plaque in an atherosclerotic lesion.

Moreover, the present invention provides the use of statins, preferably Pitavastatin for producing an agent for enhancing the effect of the compound 1, its pharmaceutically acceptable salt or a hydrate thereof on prevention of thrombus formation caused by rupture of a lipid rich plaque in an atherosclerotic lesion.

Moreover, the present invention provides a method for enhancing the effect of the compound 1, its pharmaceutically acceptable salt or a hydrate thereof on prevention of thrombus formation caused by rupture of a lipid rich plaque, including administering an effective amount of statins, preferably Pitavastatin to a patient to which an effective amount of the compound 1, its pharmaceutically acceptable salt or a hydrate thereof has been administered.

Moreover, the present invention provides a method for preventing thrombus formation caused by rupture of a lipid rich plaque by enhancing the effect of the compound 1, its pharmaceutically acceptable salt or a hydrate thereof on prevention of thrombus formation caused by rupture of a lipid rich plaque, including administering an effective amount of statins, preferably Pitavastatin to a patient to which an effective amount of the compound 1, its pharmaceutically acceptable salt or a hydrate thereof has been administered.

Moreover, the present invention provides an agent for enhancing the effect of the compound 1, its pharmaceutically acceptable salt or a hydrate thereof on prevention and/or treatment of acute coronary syndrome, acute myocardial infarction, unstable angina pectoris and/or peripheral arterial obstruction, including statins, preferably Pitavastatin.

Moreover, the present invention provides a pharmaceutical composition for enhancing the effect of the compound 1, its pharmaceutically acceptable salt or a hydrate thereof as a prophylactic and/or therapeutic agent for acute coronary syndrome, acute myocardial infarction, unstable angina pectoris and/or peripheral arterial obstruction, including statins, preferably Pitavastatin and a pharmaceutically acceptable carrier.

Moreover, the present invention provides the use of statins, preferably Pitavastatin as an agent for enhancing the effect of the compound 1, its pharmaceutically acceptable salt or a hydrate thereof as a prophylactic and/or therapeutic agent for acute coronary syndrome, acute myocardial infarction, unstable angina pectoris and/or peripheral arterial obstruction.

Moreover, the present invention provides the use of statins, preferably Pitavastatin for producing an agent for enhancing the effect of the compound 1, its pharmaceutically acceptable salt or a hydrate thereof as a prophylactic and/or therapeutic agent for acute coronary syndrome, acute myocardial infarction, unstable angina pectoris and/or peripheral arterial obstruction.

Moreover, the present invention provides a method for enhancing the effect of the compound 1, its pharmaceutically acceptable salt or a hydrate thereof as a prophylactic and/or therapeutic agent for acute coronary syndrome, acute myocardial infarction, unstable angina pectoris and/or peripheral arterial obstruction, including administering an effective amount of statins, preferably Pitavastatin to a patient to which an effective amount of the compound 1, its pharmaceutically acceptable salt or a hydrate thereof has been administered.

Moreover, the present invention provides a method for preventing and/or treating acute coronary syndrome, acute myocardial infarction, unstable angina pectoris and/or peripheral arterial obstruction by enhancing the effect of the compound 1, its pharmaceutically acceptable salt or a hydrate thereof as a prophylactic and/or therapeutic agent for acute coronary syndrome, acute myocardial infarction, unstable angina pectoris and/or peripheral arterial obstruction, including administering an effective amount of statins, preferably Pitavastatin to a patient to which an effective amount of the compound 1, its pharmaceutically acceptable salt or a hydrate thereof has been administered.

Moreover, the present invention provides an agent for enhancing the effect of statins, preferably Pitavastatin on reduction, stabilization and/or prevention of rupture of a lipid rich plaque in an atherosclerotic lesion, including the compound 1, its pharmaceutically acceptable salt or a hydrate thereof.

Moreover, the present invention provides a pharmaceutical composition for enhancing the effect of statins, preferably Pitavastatin on reduction, stabilization and/or prevention of rupture of a lipid rich plaque in an atherosclerotic lesion, including the compound 1, its pharmaceutically acceptable salt or a hydrate thereof, and a pharmaceutically acceptable carrier.

Moreover, the present invention provides the use of the compound 1, its pharmaceutically acceptable salt or a hydrate thereof as an agent for enhancing the effect of statins, preferably Pitavastatin on reduction, stabilization and/or prevention of rupture of a lipid rich plaque in an atherosclerotic lesion.

Moreover, the present invention provides the use of the compound 1, its pharmaceutically acceptable salt or a hydrate thereof for producing an agent for enhancing the effect of statins, preferably Pitavastatin on reduction, stabilization and/or prevention of rupture of a lipid rich plaque in an atherosclerotic lesion.

Moreover, the present invention provides a method for enhancing the effect of statins, preferably Pitavastatin on reduction, stabilization and/or prevention of rupture of a lipid rich plaque, including administering an effective amount of the compound 1, its pharmaceutically acceptable salt or a hydrate thereof to a patient to which an effective amount of statins, preferably Pitavastatin has been administered.

Moreover, the present invention provides a method for reduction, stabilization and/or prevention of rupture of a lipid rich plaque by enhancing the effect of statins, preferably Pitavastatin on reduction, stabilization and/or prevention of rupture of a lipid rich plaque, including administering an effective amount of the compound 1, its pharmaceutically acceptable salt or a hydrate thereof to a patient to which an effective amount statins, preferably Pitavastatin has been administered.

Moreover, the present invention provides an agent for enhancing the effect of statins, preferably Pitavastatin on prevention of thrombus formation caused by rupture of a lipid rich plaque in an atherosclerotic lesion, including the compound 1, its pharmaceutically acceptable salt or a hydrate thereof.

Moreover, the present invention provides a pharmaceutical composition for enhancing the effect of statins, preferably Pitavastatin on prevention of thrombus formation caused by rupture of a lipid rich plaque in an atherosclerotic lesion, including the compound 1, its pharmaceutically acceptable salt or a hydrate thereof, and a pharmaceutically acceptable carrier.

Moreover, the present invention provides the use of the compound 1, its pharmaceutically acceptable salt or a hydrate thereof as an agent for enhancing the effect of statins, preferably Pitavastatin on prevention of thrombus formation caused by rupture of a lipid rich plaque in an atherosclerotic lesion.

Moreover, the present invention provides the use of the compound 1, its pharmaceutically acceptable salt or a hydrate thereof for producing an agent for enhancing the effect of statins, preferably Pitavastatin on prevention of thrombus formation caused by rupture of a lipid rich plaque in an atherosclerotic lesion.

Moreover, the present invention provides a method for enhancing the effect of statins, preferably Pitavastatin on prevention of thrombus formation caused by rupture of a lipid rich plaque, including administering an effective amount of the compound 1, its pharmaceutically acceptable salt or a hydrate thereof to a patient to which an effective amount of statins, preferably Pitavastatin has been administered.

Moreover, the present invention provides a method for preventing thrombus formation caused by rupture of a lipid rich plaque by enhancing the effect of statins, preferably Pitavastatin on prevention of thrombus formation caused by rupture of a lipid rich plaque, including administering an effective amount of the compound 1, its pharmaceutically acceptable salt or a hydrate thereof to a patient to which an effective amount of statins, preferably Pitavastatin has been administered.

Moreover, the present invention provides an agent for enhancing the effect of statins, preferably Pitavastatin on prevention and/or treatment of acute coronary syndrome, acute myocardial infarction, unstable angina pectoris and/or peripheral arterial obstruction, including the compound 1, its pharmaceutically acceptable salt or a hydrate thereof.

Moreover, the present invention provides a pharmaceutical composition for enhancing the effect of statins, preferably Pitavastatin as a prophylactic and/or therapeutic agent for acute coronary syndrome, acute myocardial infarction, unstable angina pectoris and/or peripheral arterial obstruction, including the compound 1, its pharmaceutically acceptable salt or a hydrate thereof, and a pharmaceutically acceptable carrier.

Moreover, the present invention provides the use of the compound 1, its pharmaceutically acceptable salt or a hydrate thereof as an agent for enhancing the effect of statins, preferably Pitavastatin as a prophylactic and/or therapeutic agent for acute coronary syndrome, acute myocardial infarction, unstable angina pectoris and/or peripheral arterial obstruction.

Moreover, the present invention provides the use of the compound 1, its pharmaceutically acceptable salt or a hydrate thereof for producing an agent for enhancing the effect of statins, preferably Pitavastatin as a prophylactic and/or therapeutic agent for acute coronary syndrome, acute myocardial infarction, unstable angina pectoris and/or peripheral arterial obstruction.

Moreover, the present invention provides a method for enhancing the effect of statins, preferably Pitavastatin as a prophylactic and/or therapeutic agent for acute coronary syndrome, acute myocardial infarction, unstable angina pectoris and/or peripheral arterial obstruction, including administering an effective amount of the compound 1, its pharmaceutically acceptable salt or a hydrate thereof to a patient to which an effective amount of statins, preferably Pitavastatin has been administered.

Moreover, the present invention provides a method for preventing and/or treating acute coronary syndrome, acute myocardial infarction, unstable angina pectoris and/or peripheral arterial obstruction by enhancing the effect of statins, preferably Pitavastatin as a prophylactic and/or therapeutic agent for acute coronary syndrome, acute myocardial infarction, unstable angina pectoris and/or peripheral arterial obstruction, including administering an effective amount of the compound 1, its pharmaceutically acceptable salt or a hydrate thereof to a patient to which an effective amount of statins, preferably Pitavastatin has been administered.

### BRIEF DESCRIPTION OF THE DRAWINGS

FIG. 1A is an Azan-stained histopathological image of a WHHL (Watanabe Heritable Hyperlipidemic) rabbit (control).

FIG. 1B is an Azan-stained histopathological image of a WHHL rabbit (Pitavastatin 0.5 mg/kg).

FIG. 1C is an Azan-stained histopathological image of a WHHL rabbit (compound 1 10 mg/kg).

FIG. 1D is an Azan-stained histopathological image of a WHHL rabbit (Pitavastatin 0.5 mg/kg and the compound 1 10 mg/kg).

FIG. 2A is graph showing the percentage of area occupied by macrophages in an atherosclerotic lesion calculated using image analysis of the images shown in FIGS. 1A to 1D.

FIG. 2B is graph showing show the percentage of area occupied by collagen in an atherosclerotic lesion calculated using image analysis of the images shown in FIGS. 1A to 1D.

### BEST MODE FOR CARRYING OUT THE INVENTION

The present inventors have investigated the effect of combined use of statin and an ACAT inhibitor on plaque stabilization by the use of WHHL rabbits.

For the investigation of the effect of these drugs on total plasma cholesterol (TC), one or both of the compound 1 (10 mg/kg) and Pitavastatin (0.5 mg/kg) were administered to WHHL rabbits via drinking water for 16 weeks to measure the TC of each of the WHHL rabbits. The results are shown in Table 1.

TABLE 1

| | Total plasma | Inhibition |
|---|---|---|
| | cholesterol (mg/dL) | ratio |
| Drugs | Mean ± S.E | (%) |
| Control group | 646.8 ± 27.1 | |
| Pitavastatin (0.5 mg/kg) | 524.2 ± 35.6 | 19.0 |
| administration group | | |
| Compound 1 (10 mg/kg) | 648.3 ± 82.2 | — |
| administration group | | |
| Pitavastatin (0.5 mg/kg) + | 544.7 ± 36.0 | 15.8 |
| Compound 1 (10 mg/kg) | | |
| administration group | | |

While single administration group of Pitavastatin decreased TC by 19%, single administration of the compound 1 had no effect on TC because TC measured after single administration of the compound 1 was substantially the same as that of control. Further, TC measured after combined administration of Pitavastatin and the compound 1 was substantially the same as that measured after single administration of Pitavastatin.

Next, the effect of the drugs on the ratio of aortic plaque area was investigated. The results are shown in Table 2.

TABLE 2

| | Ratio of aortic | Inhibition |
|---|---|---|
| | plaque area (%) | ratio |
| Drugs | Mean ± S.E | (%) |
| Control group | 49.6 ± 5.0 | |
| Pitavastatin (0.5 mg/kg) | 47.1 ± 6.6 | 5.0 |
| administration group | | |
| Compound 1 (10 mg/kg) | 43.7 ± 4.1 | 11.9 |
| administration group | | |
| Pitavastatin (0.5 mg/kg) + | 33.1 ± 7.6 | 33.3 |
| Compound 1 (10 mg/kg) | | |
| administration group | | |

While single administration of Pitavastatin and single administration of the compound 1 decreased the ratio of aortic plaque area by 5.0% and 11.9% respectively, combined administration of Pitavastatin and the compound 1 significantly decreased the ratio of aortic plaque area by 33.3%.

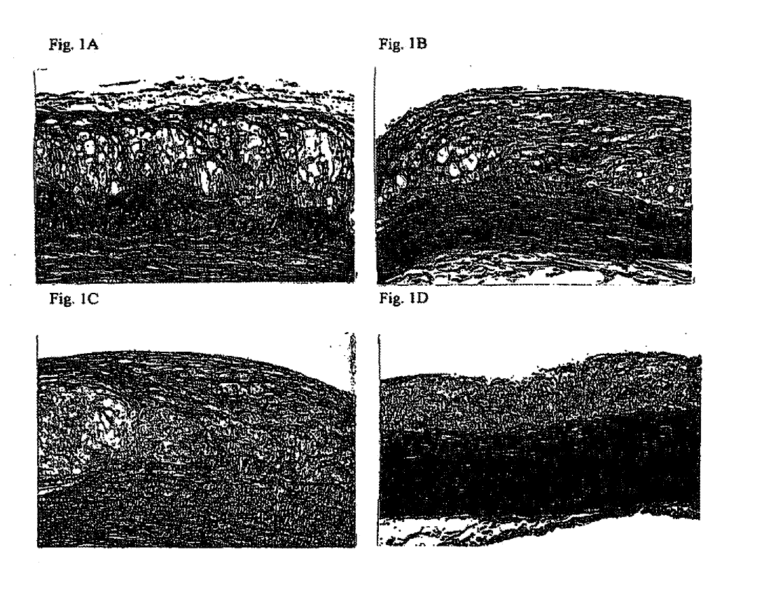

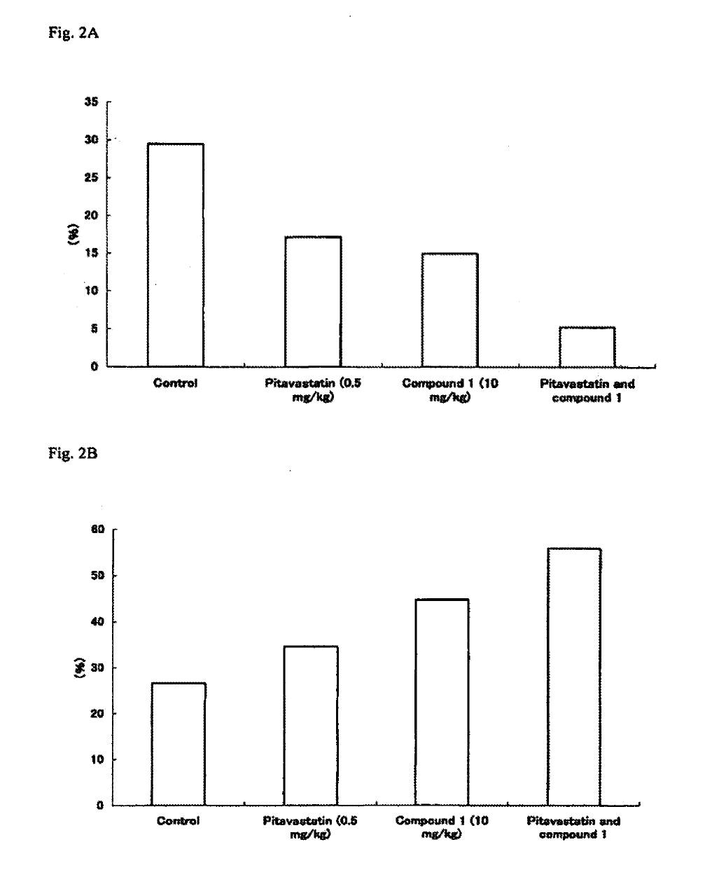

In addition to the evaluation of TC, an aorta excised from each WHHL rabbit was histopathologically examined After the completion of measurement, an aortic arch of each WHHL rabbit was immersion-fixed with 4% paraformaldehyde overnight, and was then embedded in paraffin in the usual manner to prepare a paraffin section. The paraffin section was stained with Azan stain. The thus prepared preparation was observed with a microscope, and an obtained image was analyzed with an image-analysis system software (Win Roof, Ver 5.0, MITANI CORPORATION) to determine the percentage of plaque area occupied by macrophages and the percentage of plaque area occupied by collagen by calculation. FIGS. 1A to 1D are microscope photographs of Azan-stained aortic arches of the WHHL rabbits, and FIGS. 2A and 2B are graphs which show the analysis results of the images shown in FIGS. 1A to 1D. In this regard, it is to be noted that FIG. 1A is a microscope photograph of a control group to which no drug was administered, FIG. 1B is a microscope photograph of a group to which Pitavastatin was administered in single, FIG. 1C is a microscope photograph of a group to which the compound 1 was administered in single, and FIG. 1D is a microscope photograph of a group to which Pitavastatin and the compound 1 were administered in combination.

In the case of the control group to which no drug was administered, accumulation of foam cells derived from macrophages and a little amount of collagen were observed from the surface of a thickened intimal lesion to the middle portion of the lesion (see FIG. 1A), and the percentage of plaque area occupied by macrophages was 29.5%. Collagen was observed from the middle portion to the lower layer of the lesion and around macrophages (see FIG. 1A), and the percentage of plaque area occupied by collagen was 26.5%. In both cases of single administration of Pitavastatin and single administration of the compound 1, macrophage foam cell formation was inhibited as compared to the control group (see FIGS. 1B and 1C respectively), and the percentages of plaque area occupied by macrophages were decreased to 17.1% and 14.9%, respectively. In addition, in both cases of single administration of Pitavastatin and single administration of the compound 1, collagen was increased in plaques, and the percentages of plaque area occupied by collagen were 34.7% and 44.8%, respectively. In the case of combined administration of Pitavastatin and the compound 1, macrophage foam cell formation was more significantly inhibited as compared to single administration of Pitavastatin or the compound 1, and the percentage of plaque area occupied by macrophages was only 5.3%. In addition, the percentage of plaque area occupied by collagen was significantly increased to 56.1%. The microscope photograph of FIG. 1D indicates that combined administration of Pitavastatin and the compound 1 more significantly stabilized plaques as compared to single administration of Pitavastatin or the compound 1.

FIGS. 2A and 2B are graphs which show the percentage of area occupied by macrophages in an atherosclerotic lesion and the percentage of area occupied by collagen in an atherosclerotic lesion, respectively determined using image analysis of the images shown in FIGS. 1A to 1D. In FIGS. 2A and 2B, “Control” represents a control group to which no drug was administered, “Pitavastatin (0.5 mg/kg)” represents a group to which Pitavastatin was administered singly, “Compound 1 (10 mg/kg)” represents a group to which the compound 1 was administered singly, and “Pitavastatin and compound 1” represents a group to which Pitavastatin and the compound 1 were administered in combination. As shown in FIGS. 2A and 2B, the control group had the highest percentage of area occupied by macrophages in an atherosclerotic lesion and the lowest percentage of area occupied by collagen in an atherosclerotic lesion among these four groups. Further, single administration of Pitavastatin or the compound 1 had the tendency to decrease the percentage of area occupied by macrophages in an atherosclerotic lesion and increase the percentage of area occupied by collagen in an atherosclerotic lesion as compared to the control group, that is, single administration of Pitavastatin or the compound 1 had the effect of stabilizing plaques to some extent. Furthermore, combined administration of Pitavastatin and the compound 1 more significantly decreased the percentage of area occupied by macrophages in an atherosclerotic lesion and increased the percentage of area occupied by collagen in an atherosclerotic lesion, that is, combined administration group of Pitavastatin and the compound 1 had the effect of significantly stabilizing plaques.

As described above, Pitavastatin not only decreased TC levels of the WHHL rabbits and slightly reduced plaques thereof but also stabilized the plaques by decreasing macrophages and increasing collagen in the plaques. The compound 1 also had the effect of reducing plaques, decreasing macrophages and increasing collagen. However, from the fact that single administration of the compound 1 did not decrease TC, it can be considered that the compound 1 directly acted on macrophages in plaques to inhibit ACAT so that the macrophage foam cell formation was inhibited. Further, combined administration of Pitavastatin and the compound 1 not only more significantly reduced plaques but also more significantly decreased the percentage of area occupied by macrophages in an atherosclerotic lesion and increased the percentage of area occupied by collagen in an atherosclerotic lesion as compared to single administration group of Pitavastatin or the compound 1. This indicates that combined administration of Pitavastatin and the compound 1 has a more significant effect on enhancement of plaque stabilization.

As described above, the present invention relates to a method for reduction of a lipid rich plaque, a method for stabilization of a lipid rich plaque, and/or a method for prevention of rupture of a lipid rich plaque. These methods according to the present invention can be employed singly or in combination of two or more. Since rupture of a lipid rich plaque is often prevented by stabilizing a lipid rich plaque, it is preferred that the method for stabilization of a lipid rich plaque and the method for prevention of rupture of a lipid rich plaque be employed together. In this specification, there is a case where the use of the compound 1, its pharmaceutically acceptable salt or a hydrate thereof and statins such as Pitavastatin relating to these methods according to the present invention to be employed singly or in combination of two or more of them, for example relating to (1) a method for reduction of a lipid rich plaque, (2) a method for stabilization of a lipid rich plaque, (3) a method for prevention of rupture of a lipid rich plaque, (4) a method for reduction of a lipid rich plaque and stabilization of a lipid rich plaque, (5) a method for reduction of a lipid rich plaque and prevention of rupture of a lipid rich plaque, (6) a method for stabilization of a lipid rich plaque and prevention of rupture of a lipid rich plaque, or (7) a method for reduction of a lipid rich plaque, stabilization of a lipid rich plaque, and prevention of rupture of a lipid rich plaque, is simply referred to as reduction, stabilization and/or prevention of rupture of a lipid rich plaque.

Specifically, the present invention provides a method for reduction, stabilization and/or prevention of rupture of a lipid rich plaque, including administering an effective amount of a pharmaceutical composition containing an active ingredient composed of one or more of the compound 1, its pharmaceutically acceptable salt and a hydrate thereof together with Pitavastatin to a patient with a lipid rich plaque. Further, the present invention also provides a pharmaceutical composition which can enhance the effect of reduction, stabilization and/or prevention of rupture of a lipid rich plaque, including an active ingredient composed of one or more of the compound 1, its pharmaceutically acceptable salt and a hydrate thereof, Pitavastatin, and a pharmaceutically acceptable carrier.

Combined administration of the compound 1 according to the present invention, its pharmaceutically acceptable salt or a hydrate thereof and Pitavastatin more significantly reduces a lipid rich plaque, increases collagen, and decreases the percentage of plaque area occupied by macrophages as compared to single administration of any one of them, and therefore can enhance the stabilization of a lipid rich plaque, prevent rupture of a lipid rich plaque, and enhance the effect of preventing thrombus formation. Therefore, the present invention also provides a prophylactic and/or therapeutic agent for various thrombosis-related diseases such as thrombosis, acute coronary syndrome, acute myocardial infarction, unstable angina pectoris, and peripheral arterial obstruction, a pharmaceutical composition thereof, a prophylactic and/or therapeutic method using the prophylactic and/or therapeutic agent or the pharmaceutical composition, and the use of the compound 1, its pharmaceutically acceptable salt or a hydrate thereof and Pitavastatin for producing the prophylactic and/or therapeutic agent or the pharmaceutical composition.

Further, since statins such as Pitavastatin significantly enhance the effect of the compound 1, its pharmaceutically acceptable salt or a hydrate thereof on reduction, stabilization and/or prevention of rupture of a lipid rich plaque, the present invention also provides an agent for enhancing the pharmacological effect of the compound 1, its pharmaceutically acceptable salt or a hydrate thereof on reduction, stabilization and/or prevention of rupture of a lipid rich plaque, including statins, preferably Pitavastatin.

Furthermore, since the compound 1, its pharmaceutically acceptable salt or a hydrate thereof significantly enhances the effect of statins such as Pitavastatin on reduction, stabilization and/or prevention of rupture of a lipid rich plaque, the present invention also provides an agent for enhancing the pharmacological effect of statins, preferably Pitavastatin on reduction, stabilization and/or prevention of rupture of a lipid rich plaque, including the compound 1, its pharmaceutically acceptable salt or a hydrate thereof.

The compound 1, its pharmaceutically acceptable salt or a hydrate thereof and Pitavastatin to be used in the present invention can be separately formulated into different preparations by, for example, the following methods, and such different pharmaceutical preparations can be used simultaneously, separately or sequentially. Alternatively, an effective amount of the compound 1, its pharmaceutically acceptable salt or a hydrate thereof and an effective amount of Pitavastatin may be mixed in an appropriate ratio to formulate them into a single dosage form. Examples of such preparations include oral agents and parenteral agents such as injections, suppositories, ointments, and patches. These preparations can be produced by a formulation method well known to those skilled in the art using a pharmaceutically acceptable carrier as a component selected according to a dosage form. In the present invention, a pharmaceutically acceptable salt of the compound 1 or a hydrate of the compound 1 or its pharmaceutically acceptable salt can also be used, and these pharmaceutically acceptable salt and hydrate can be obtained in the usual manner. Here, examples of an acid for forming a pharmaceutically acceptable salt, for example, an acid adduct salt include: inorganic acids such as hydrochloric acid, sulfuric acid, nitric acid, phosphoric acid, hydrobromic acid, and hydriodic acid; and organic acids such as acetic acid, lactic acid, succinic acid, tartaric acid, malic acid, maleic acid, fumaric acid, citric acid, ascorbic acid, methansulfonic acid, besylic acid, and toluenesulfonic acid. As an active ingredient, the compound 1 according to the present invention, its pharmaceutically acceptable salt and a hydrate thereof can be used singly or in combination of two or more of them.

Oral solid preparations such as tablets, coated tablets, granules, powders, and capsules can be produced by, for example, adding an excipient, and if necessary, a binder, a disintegrator, a lubricant, a coloring agent, a corrigent or a flavoring agent to the compound 1, its pharmaceutically acceptable salt or a hydrate thereof and/or Pitavastatin in the usual manner. As such additives, those generally used in this field can be employed. Examples of an excipient include lactose, sucrose, sodium chloride, glucose, starch, calcium carbonate, kaolin, microcrystalline cellulose, and silicic acid. Examples of a binder include water, ethanol, propanol, simple syrup, glucose solution, starch solution, gelatin solution, carboxymethyl cellulose, hydroxypropyl cellulose, hydroxypropyl starch, methyl cellulose, ethyl cellulose, shellac, calcium phosphate, and polyvinyl pyrrolidone. Examples of a disintegrator include dry starch, sodium alginate, agar powder, sodium acid carbonate, calcium carbonate, sodium lauryl sulfate, stearic acid monoglyceride, and lactose. Examples of a lubricant include purified talc, stearic acid salts, borax, and polyethylene glycol. Examples of a corrigent include sucrose, orange peel, citric acid, and tartaric acid.

Oral liquid preparations such as liquids for internal use, syrups, and elixirs can be produced by, for example, adding, if necessary, a corrigent, a buffering agent, a stabilizer or a flavoring agent to the compound 1, its pharmaceutically acceptable salt or a hydrate thereof and/or Pitavastatin in the usual manner. In this case, as a corrigent, the same corrigents as mentioned above can be used. An example of a buffering agent includes sodium citrate. Examples of a stabilizer include tragacanth, gum arabic, and gelatin.

Injections such as subcutaneously-administered injections, intramuscularly-administered injections, and intravenously-administered injections can be produced by, for example, adding, if necessary, a pH regulator, a buffering agent, a stabilizer, a tonicity agent or a local anesthetic agent to the compound 1, its pharmaceutically acceptable salt or a hydrate thereof and/or Pitavastatin in the usual manner. In this case, examples of a pH regulator and a buffering agent include sodium citrate, sodium acetate, and sodium phosphate. Examples of a stabilizer include sodium pyrosulfite, EDTA, thioglycolic acid, and thiolactic acid. Examples of a local anesthetic agent include procaine hydrochloride and lidocaine hydrochloride. Examples of a tonicity agent include sodium chloride and glucose.

The compound 1, its pharmaceutically acceptable salt or a hydrate thereof and/or Pitavastatin can be formulated into other dosage forms according to well-known methods.

The thus produced different pharmaceutical preparations can be separately packaged. In this case, each of the pharmaceutical preparations is taken out from a package when administered. Alternatively, these different pharmaceutical preparations may be packaged in a form suitable for combined administration per one dosage.

The pharmaceutical composition according to the present invention having the effect of significantly reducing, stabilizing, and preventing rupture of a lipid rich plaque can be effectively used not only as a prophylactic and/or therapeutic drug for reduction, stabilization, and prevention of rupture of a lipid rich plaque in an atherosclerotic lesion but also as a prophylactic and/or therapeutic drug for preventing thrombus formation caused by rupture of a lipid rich plaque, a prophylactic and/or therapeutic drug for acute coronary syndrome, a prophylactic and/or therapeutic drug for acute myocardial infarction, a prophylactic and/or therapeutic drug for unstable angina pectoris, and a prophylactic and/or therapeutic drug for peripheral arterial obstruction.

Although the dose of the pharmaceutical composition according to the present invention having the effect of enhancing plaque stabilization varies depending on, for example, the body weight, age, sex, and symptom of a patient, dosage form, and the number of doses, a daily dose of the compound 1, its pharmaceutically acceptable salt or a hydrate thereof for an adult is generally 0.01 to 1,000 mg, preferably 0.1 to 100 mg, which is preferably administered orally or parenterally in a single dose or several divided doses. A daily dose of Pitavastatin for an adult is generally 0.01 to 16 mg, preferably 0.05 to 4 mg, more preferably 0.1 to 2 mg, which is preferably administered orally in a single dose or several divided doses. In a case where the compound 1, its pharmaceutically acceptable salt or a hydrate thereof and Pitavastatin are separately formulated into different preparations, these preparations are administered at the same time or administered at an interval of 15 minutes to 24 hours.

### EXAMPLES

Hereinbelow, the present invention will be described in detail with reference to the following examples, but the present invention is not limited thereto.

A WHHL rabbit was discovered by Dr. Yoshio Watanabe, a former medical professor at Kobe University in 1973, and was established as a strain. The WHHL rabbit is a model animal which naturally develops hypercholesterolemia and arteriosclerosis. Using such WHHL rabbits, the effect of the drugs on plaque stabilization was examined according to the following method.

(1) Test Method

1. Test Animal

Male homozygous WHHL rabbits (Kitayama Labes, Nagano) were purchased from Oriental Yeast (Tokyo), and WHHL rabbits aged about 4 months were used for experiment. For control, single administration of Pitavastatin, single administration of the compound 1, combined administration of Pitavastatin and the compound 1, 8 WHHL rabbits, 7 WHHL rabbits, 3 WHHL rabbits, and 4 WHHL rabbits were used, respectively.

2. Test Drugs, Preparation and Administration Methods of Test Drugs, and Length of Dosing Period

Pitavastatin was synthesized by Nissan Chemical Industries, Ltd. (Tokyo) and was supplied therefrom. In the case of administration of Pitavastatin, 0.5 mg/kg of Pitavastatin dissolved in 400 mL (which is an average amount of drinking water per rabbit a day) of drinking water was administered to each rabbit. In the case of administration of the compound 1, 10 mg/kg (in terms of free base) of the compound 1 dissolved in 400 mL (which is an average amount of drinking water per rabbit a day) of ion-exchange water was administered to each rabbit. One or both of the drugs were administered for 16 weeks.

3. Observation and Test Methods

A. Total Plasma Cholesterol (TC) Level

In the next morning after last administration, each of the rabbits was placed in a restraining box, and the ear of the rabbit was sterilized with an alcohol. Then, about 1 mL of blood was collected from an ear vein with a syringe containing EDTA (final concentration, 0.1%). The collected blood was centrifuged at 4,200×g for 5 minutes to fractionate plasma. TC was measured using cholesterol E-test Wako (by a cholesterol oxidaseΩDAOS method).

B. Measurement of Ratio of Aortic Plaque Area and Pathological Study

The rabbit was perfused at a perfusion pressure of 120 mmH₂O with physiological saline injected from a cardiac apex for about 5 minutes. Further, the rabbit was perfused with 4% paraformaldehyde in the same manner as described above to fix an aorta. The aorta was excised from the rabbit, and fat and connective tissue attached to the aorta were separated. An incision was made along the lesser curvature of the aorta, and then the greater curvature of the aorta was partially incised. The aorta was sandwiched between plastic films in such a manner that the intimal side of the aorta was observed, and a macro image was taken (CAMEDIA E-10, manufactured by Olympus Corporation). Based on the image, plaque area (that is, the area of lipid deposition) and aortic intimal surface area were analyzed by an image analyzer (Win Roof Ver. 5.0, manufactured by MITANI CORPORATION). The ratio of aortic plaque area of each of the rabbits was obtained using the formula:

plaque area/aortic intimal surface area×100

After the completion of measurement, an aortic arch of each of the rabbits was immersed in 4% paraformaldehyde overnight for fixation, and was then embedded in paraffin in the usual manner to prepare a paraffin section. The paraffin section was stained with Azan. The thus prepared preparation was observed with a microscope, and an obtained image was analyzed by an image-analysis system software (Win Roof Ver. 5.0, MITANI CORPORATION) to determine the percentage of plaque area occupied by macrophages and the percentage of plaque area occupied by collagen.

4. Statistical Analysis and Data Processing Methods

The results of TC and the ratio of aortic plaque area were expressed as average±standard error. Significance test between the control group and the drug administration groups were detected using Dunnett's test. Histopathological findings were evaluated in terms of the percentage of plaque area occupied by macrophages and the percentage of plaque area occupied by collagen.

(2) Results

A. TC Level

The effect of Pitavastatin and the compound 1 on TC is shown in Table 1. While Pitavastatin decreased TC by 19.0%, the compound 1 had no effect on TC. TC measured after combined administration of Pitavastatin and the compound 1 was substantially the same as that measured after single administration of Pitavastatin.

B. Ratio of Aortic Plaque Area

The effect of Pitavastatin and the compound 1 on the ratio of aortic plaque area is shown in Table 2. Single administration group of Pitavastatin and single administration of the compound 1 decreased the ratio of aortic plaque area by 5.0% and 11.9%, respectively. Combined administration of Pitavastatin and the compound 1 significantly decreased the ratio of aortic plaque area by 33.3%.

C. Histopathological Findings

FIGS. 1A to 1D are microscope photographs of Azan-stained aortic arches of the WHHL rabbits, and FIGS. 2 A and 2B are graphs which show the analysis results of the images shown in FIGS. 1A to 1D.

In the case of the control group, accumulation of foam cells derived from macrophages was observed from the surface of a thickened intimal lesion to the middle portion of the lesion (see FIG. 1A). The percentage of plaque area occupied by macrophages was 29.5%, which was the highest among the four groups. Collagen was observed from the middle portion to the lower layer of the lesion and around macrophages (see FIG. 1A). The percentage of plaque area occupied by collagen was 26.5%, which was the lowest among the four groups. The plaques observed in the control group were unstable because they were poor in fibrous components and rich in foam cells derived from macrophages. In both cases of single administration of Pitavastatin and single administration of the compound 1, macrophage foam cell formation was inhibited as compared to the control group (see FIGS. 1B and 1C, respectively), and the percentages of plaque area occupied by macrophages were decreased to 17.1% and 14.9%, respectively.

In addition, in both cases of single administration of Pitavastatin and single administration of the compound 1, collagen was increased in plaques, and the percentages of plaque area occupied by collagen were 34.7% and 44.8%, respectively, which were higher than that of the control group. The plaques observed after single administration of Pitavastatin or the compound 1 were stabilized. In the case of combined administration of Pitavastatin and the compound 1, macrophage foam cell formation was more significantly inhibited as compared to single administration of Pitavastatin or the compound 1, and the percentage of plaque area occupied by macrophages was only 5.3%. In addition, the percentage of plaque area occupied by collagen was 56.1%, which was the highest among the four groups. The plaques observed after combined administration of Pitavastatin and the compound 1 were further stabilized as compared to those observed after single administration of Pitavastatin or the compound 1.

As described above, Pitavastatin not only decreased TC levels of the WHHL rabbits and reduced plaque area but also stabilized plaques of the WHHL rabbits by decreasing macrophages and increasing collagen in the plaques. The compound 1 also had the effect of reducing plaque area, decreasing macrophages and increasing collagen. From the fact that single administration of the compound 1 did not decrease TC, it can be considered that the compound 1 directly acted on macrophages in plaques to inhibit ACAT so that the macrophage foam cell formation was inhibited. Further, combined administration of Pitavastatin and the compound 1 more significantly produced qualitative change such as decrease in macrophages and increase in collagen as well as quantitative change such as reduction in plaque area as compared to single administration group of Pitavastatin or the compound 1. That is, combined administration of Pitavastatin and the compound 1 had a strong effect on enhancement of plaque stabilization.

### INDUSTRIAL APPLICABILITY

According to the present invention, it is possible to provide a pharmaceutical composition for reduction, stabilization and/or prevention of rupture of a lipid rich plaque in an atherosclerotic lesion, a agent for preventing and/or treating thrombus formation caused by rupture of a lipid rich plaque, and a prophylactic and/or therapeutic drug for acute coronary syndrome, acute myocardial infarction, unstable angina pectoris, and/or peripheral arterial obstruction. Specifically, it is possible to provide a drug, a pharmaceutical preparation or a pharmaceutical composition having an enhanced effect on reduction, stabilization and/or prevention of rupture of a lipid rich plaque by reducing plaque area in a plaque lesion, inhibiting accumulation of macrophages, and increasing collagen due to combined administration of an effective amount of 2-[4-[2-(benzimidazole-2-ylthio)ethyl]piperazin-1-yl]-N-[2,4-bis(methylthio)-6-methyl-3-p yridyl]acetamide, its pharmaceutically acceptable salt or a hydrate thereof and Pitavastatin, or a drug, a pharmaceutical preparation or a pharmaceutical composition for enhancing such an effect.

What is claimed is:

1. A pharmaceutical composition comprising:
2-[4-[2-(benzimidazole-2-ylthio)ethyl]piperazin-1-yl]-[2,4-bis(methylthio)-6-met hyl-3-pyridyl]acetamide, its pharmaceutically acceptable salt or a hydrate thereof, Pitavastatin, and
a pharmaceutically acceptable carrier,
wherein the composition is a single pharmaceutical preparation containing these active ingredients.

2. A method for preventing rupture of a lipid rich plaque in an atherosclerotic lesion by reduction of area of a lipid rich plaque and/or stabilization of the lipid rich plaque, comprising:
simultaneously administering, or separately administering with interval of time an effective amount of 2-[4-[2-(benzimidazole-2-ylthio)ethyl]piperazin-1-yl]-N-[2,4-bis(methylthio)-6-methyl-3-p yridyl]acetamide, its pharmaceutically acceptable salt or a hydrate thereof and an effective amount of Pitavastatin to a patient in need of the prevention of rupture of a lipid rich plaque in an atherosclerotic lesion.

3. The method according to claim 2, wherein an effective amount of 2-[4-[2-(benzimidazole-2-ylthio)ethyl]piperazin-1-yl]-N-[2,4-bis(methylthio)-6-methyl-3-p yridyl]acetamide, its pharmaceutically acceptable salt or a hydrate thereof and an effective amount of Pitavastatin are administered as a single pharmaceutical preparation containing these active ingredients.

4. The method according to claim 2, wherein Pitavastatin is administered after 2-[4-[2-(benzimidazole-2-ylthio)ethyl]piperazin-1-yl]-N-[2,4-bis(methylthio)-6-methyl-3-p yridyl]acetamide, its pharmaceutically acceptable salt or a hydrate thereof is administered.

5. The method according to claim 2, wherein 2-[4-[2-(benzimidazole-2-ylthio)ethyl]piperazin-1-yl]-N-[2,4-bis(methylthio)-6-methyl-3-p yridyl]acetamide, its pharmaceutically acceptable salt or a hydrate thereof is administered after Pitavastatin is administered.

6. A method for preventing thrombus formation which is caused by the rupture of a lipid rich plaque, comprising:
simultaneously administering, or separately administering with interval of time an effective amount of 2-[4-[2-(benzimidazole-2-ylthio)ethyl]piperazin-1-yl]-N-[2,4-bis(methylthio)-6-methyl-3-p yridyl]acetamide, its pharmaceutically acceptable salt or a hydrate thereof and an effective amount of Pitavastatin to a patient in need of the prevention of thrombus formation caused by rupture of the lipid rich plaque.

7. The method according to claim 6, wherein an effective amount of 2-[4-[2-(benzimidazole-2-ylthio)ethyl]piperazin-1-yl]-N-[2,4-bis(methylthio)-6-methyl-3-p yridyl]acetamide, its pharmaceutically acceptable salt or a hydrate thereof and an effective amount of Pitavastatin are administered as a single pharmaceutical preparation containing these active ingredients.

8. The method according to claim 6, wherein Pitavastatin is administered after 2-[4-[2-(benzimidazole-2-ylthio)ethyl]piperazin-1-yl]-N-[2,4-bis(methylthio)-6-methyl-3-p yridyl]acetamide, its pharmaceutically acceptable salt or a hydrate thereof is administered.

9. The method according to claim 6, wherein 2-[4-[2-(benzimidazole-2-ylthio)ethyl]piperazin-1-yl]-N-[2,4-bis(methylthio)-6-methyl-3-p yridyl]acetamide, its pharmaceutically acceptable salt or a hydrate thereof is administered after Pitavastatin is administered.

10. A prophylactic method for acute coronary syndrome, acute myocardial infarction, unstable angina pectoris, and/or peripheral arterial obstruction, comprising:
simultaneously administering, or separately administering with interval of time an effective amount of 2-[4-[2-(benzimidazole-2-ylthio)ethyl]piperazin-1-yl]-N-[2,4-bis(methylthio)-6-methyl-3-p yridyl]acetamide, its pharmaceutically acceptable salt or a hydrate thereof and an effective amount of Pitavastatin to a patient in need of the prophylactic treatment for acute coronary syndrome, acute myocardial infarction, unstable angina pectoris, and/or peripheral arterial obstruction.

11. The prophylactic method according to claim 10, wherein an effective amount of 2-[4-[2-(benzimidazole-2-ylthio)ethyl]piperazin-1-yl]-N-[2,4-bis(methylthio)-6-methyl-3-p yridyl]acetamide, its pharmaceutically acceptable salt or a hydrate thereof and an effective amount of Pitavastatin are administered as a single pharmaceutical preparation containing these active ingredients.

12. The prophylactic method according to claim 10, wherein Pitavastatin is administered after 2-[4-[2-(benzimidazole-2-ylthio)ethyl]piperazin-1-yl]-N-[2,4-bis(methylthio)-6-methyl-3-p yridyl]acetamide, its pharmaceutically acceptable salt or a hydrate thereof is administered.

13. The prophylactic method according to claim 10, wherein 2-[4-[2-(benzimidazole-2-ylthio)ethyl]piperazin-1-yl]-N-[2,4-bis(methylthio)-6-methyl-3-p yridyl]acetamide, its pharmaceutically acceptable salt or a hydrate thereof is administered after Pitavastatin is administered.
